(19) United States
(12) Patent Application Publication
(10) Pub. No.: US 2005/0215924 A1
(43) Pub. Date: Sep. 29, 2005

(54) Noninvasive device for collecting bio-specimen on gynaecology

(76) Inventors: Yi-Feng Kao, Sanchong City (TW); Ching-Yu Lin, Yilan City (TW); Peter Chan, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/084,094

(22) Filed: Mar. 21, 2005

(30) Foreign Application Priority Data
Mar. 23, 2004 (TW) 093107720

(51) Int. Cl. A61B 5/00; B65D 81/00; A61F 13/15; A61F 13/20; A61B 10/00

(52) U.S. Cl. 600/573; 604/358; 604/385.01; 604/385.14

(57) ABSTRACT

The present invention discloses a noninvasive collection device for collecting bio-specimens of female, which comprises an absorbing layer and a supporting layer, wherein the absorbing layer is used for collecting bio-specimens, such as secretions, cells, and tissues from the genital regions. The supporting layer is used to fix the mentioned noninvasive collection device onto a substrate, such as: sanitary napkin. Furthermore, the noninvasive collection device can be employed in menstrual cycle or in ordinary days. Moreover, the used collection device can be stored in containers for transportation, gene extraction, and subsequent analysis.

### BACKGROUND OF THE INVENTION

1. Field of the Invention

The present invention is generally related to a sampling device, and more particularly to a noninvasive collection device for collecting bio-specimens of female.

2. Description of the Prior Art

Pelvic examinations and smear test are common medical technologies associated with female diseases. Pelvic examination is performed by inserting a speculum into examinee's vagina and holding open the walls of the vagina, so that the doctor can inspect and palpate the vulva, vaginal, and cervix, and abnormal tissues would be found. On the other hand, Pap smear test is the most popular example of smear test. The procedure of Pap smear test is as following: First, a speculum is inserted into examinee's vagina and holding open the walls of the vagina. Next, cervix brush is used to scrape some cells from the surface and the inside of the cervix. These cells are then smeared onto glass slides, sprayed with a fixative to fix the cells, and then sent to a laboratory for examination under a microscope.

However, according to clinical practice, most Asian women find the Pap smear and pelvic examinations embarrassing, and false positive or false negative results of Pap smear can happen and caused by wrong judgment or incorrect sampling procedure. Furthermore, it usually takes 1˜2 weeks to complete the Pap smear procedure (from sampling to getting result), and if the procedure is failed, the examinee should take another Pap smear procedure after 4˜6 weeks. On the other hand, Smear tests can not detect abnormal cells caused by Human Papilloma Virus (HPV) in their early stage, but other medical technologies can detect the early developing abnormal cells. Besides, there are still many infection agents can not be detected by smear test, and the accuracy of smear test need to be improved.

In early 1990s, there is reported a HIV screening method by analyzing used cotton balls of patients. In 2002, Tommy R. Tong et al. disclosed another HIV screening method by polymerase chain reaction (PCR) of menstrual blood or secretion. Human papilloma virus was successfully detected by PCR in menstrual blood or vaginal discharge collected in sanitary napkins in 100% of examinees. On the other hand, Polymerase Chain Reaction (PCR) of menstrual blood can be also applied in Chlamydia infection screen or other Sexually Transmitted Disease (STD) screen.

To solve the above-mentioned problems associated with the common medical technologies about female diseases, a new collection device is still needed corresponding to broaden the scope of screening and to improve accuracy.

### SUMMARY OF THE INVENTION

In accordance with the present invention, one object of the present invention is to provide a new noninvasive collection device for collecting bio-specimens of female is provided that substantially overcomes the drawbacks of the above problems mentioned from the conventional system. The mentioned collection device can be temporary fixed onto a noninvasive female sanitary utensil, such as: sanitary napkin, pantyliner, or other nursing products. Therefore, the collection device is a “noninvasive” collection device. After self-sampling, the used collection device can be plucked and transported for subsequent analysis.

Another object of this invention is to provide a noninvasive collection device. The noninvasive collection device comprises a absorbing part to collect secretions, cells, and tissues from the genital regions for disease detecting, disease surveillance, investigation of related genes or other bio-technology tests.

Accordingly, the present invention discloses a noninvasive collection device, which comprises an absorbing layer and a supporting layer. The absorbing layer is located on one side of the supporting layer, and a fixing layer is located on another side of the supporting layer. The mentioned fixing layer can fix the noninvasive collection device onto a noninvasive sanitary utensil. Furthermore, after self-collecting step, the used collection device can be plucked from the noninvasive sanitary utensil for further analysis. Moreover, the used collection device can be stored in a container for transportation and subsequent bio-analysis. The mentioned container comprises centrifuge tube and test tube. The centrifuge tube is directly used in centrifuge separation; so that samples can be separated from the absorbing layer more easily. Other analysis method, like gene extraction, can be also achieved more conveniently by centrifuge tube.

Comparing to known medical technologies associated with female diseases, this invention gives facilities for home-based sample collection, and renders users conveniently to mail or transport samples to medical laboratories for disease detecting, disease surveillance, investigation of related genes or other bio-technology tests. Because the self-sampling procedure provokes less discomfort, pain, and embarrassment than pelvic examinations or Pap smear testing, the present invention provides a better option for disease screening.

### BRIEF DESCRIPTION OF THE DRAWINGS

FIG. 1A is a schematic diagram of a noninvasive collection device in accordance with the present invention;

FIG. 1B is a side view of FIG. 1A;

FIG. 2 is an exploration view in accordance with the present invention;

FIG. 3A and FIG. 3B are schematic diagrams of the noninvasive sanitary utensil combined with the noninvasive collection device in accordance with the present invention; and

FIG. 4A and FIG. 4B are schematic diagrams about storage of the noninvasive collection device in accordance with the present invention.

### DESCRIPTION OF THE PREFERRED EMBODIMENTS

Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

Furthermore, the scales of all elements in this specification are not drawn according to real elements. Besides, some elements are shown dramatically to give better understandings of the present invention.

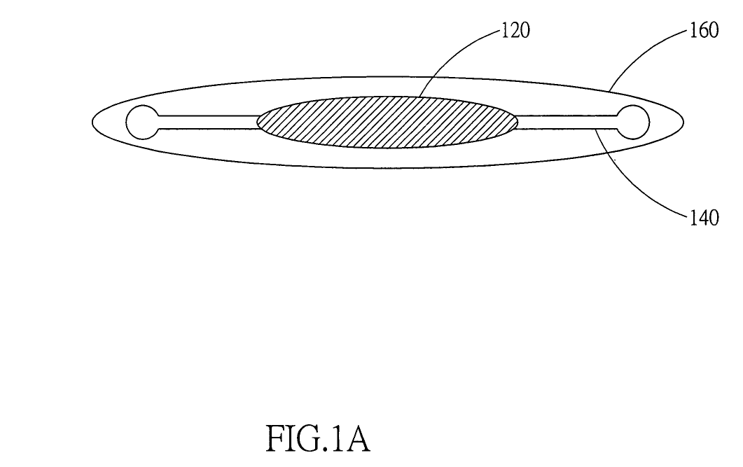

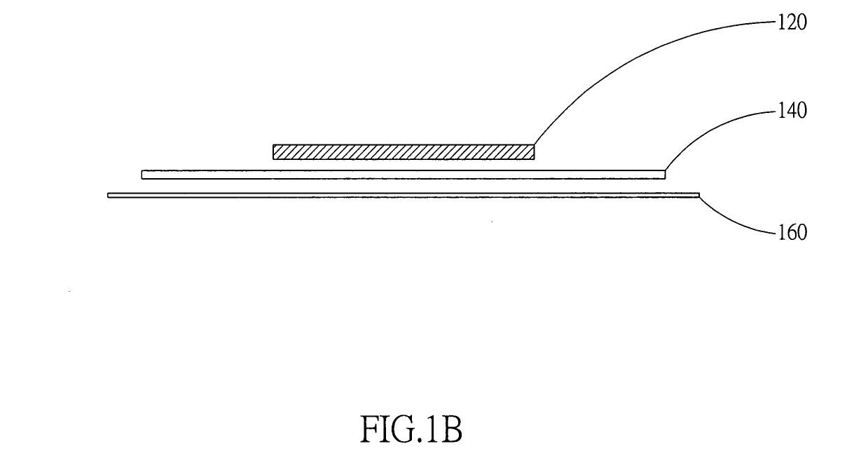

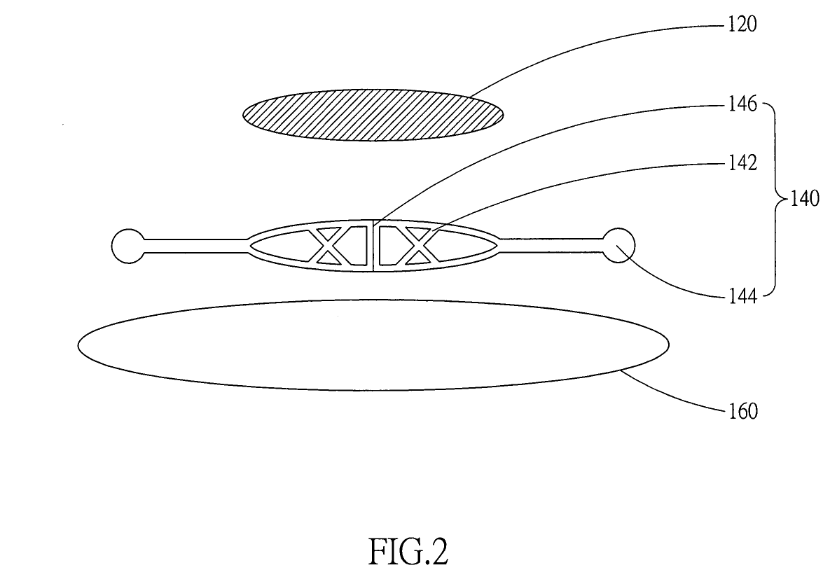

Referring to FIG. 1A and 1B, in a preferred embodiment of the present invention, a noninvasive collection device is disclosed for collecting bio-specimens of female, wherein the device comprises an absorbing layer 120 and a supporting layer 140. FIG. 2 is an exploration view of a preferred noninvasive collection device. Referring to FIG. 2, the absorbing layer 120 is fixed onto the supporting layer 140, wherein the material of the absorbing layer 120 is absorbent. Furthermore, the absorbing layer 120 can be glued onto the supporting layer 140, or through other fixing methods known by skilled persons in this art.

In a preferred example of this embodiment, the supporting layer 140 further comprises a porous part 142, and the absorbing layer 120 can be fixed onto the porous part 142. The supporting layer 140 can be glued onto the noninvasive sanitary utensil, or through other fixing methods, wherein the noninvasive sanitary utensil is sanitary napkin, pantyliner, or other nursing products. Furthermore, an adhesive layer (not shown in FIG. 2) can be provided below said supporting layer, so that the supporting layer 140 can be glued onto the mentioned noninvasive sanitary utensil. Additionally, as shown in FIG. 2, the noninvasive collection device further comprises a protective sheet 160 outside the supporting layer 140, so as to provide the mentioned noninvasive collection device with ultra portability and easy transportation.

Referring to FIG. 2, the supporting layer 140 further comprises at least one pulling element, wherein two pulling elements 144 are preferred, and two pulling elements 144 are oppositely located at the supporting layer 140. The user can pluck the supporting layer 140 and the absorbing layer 120 by use of the pulling elements 144 after self-collecting bio-specimens. Moreover, the supporting layer 140 further comprises a folding line 146, as shown in FIG. 2, and the folding line 146 is located in the middle of the supporting layer 140. The folding line 146 enables the user easily to fold the plucked supporting layer 140 and absorbing layer 120, and then the used collection device can be stored in test tube or other container for transportation and subsequent analysis.

For further descriptions of the present invention, there is provided a procedure of how to use the mentioned noninvasive collection device. First, the user fixes the noninvasive collection device onto a noninvasive sanitary utensil for collecting bio-specimens of her. The bio-specimens comprise secretions, cells, and tissues from the genital regions. Furthermore, the noninvasive collection device can be employed in menstrual cycle or in ordinary days. After self-collecting step, the used collection device can be plucked from the noninvasive sanitary utensil and stored in a container for transportation and subsequent analysis.

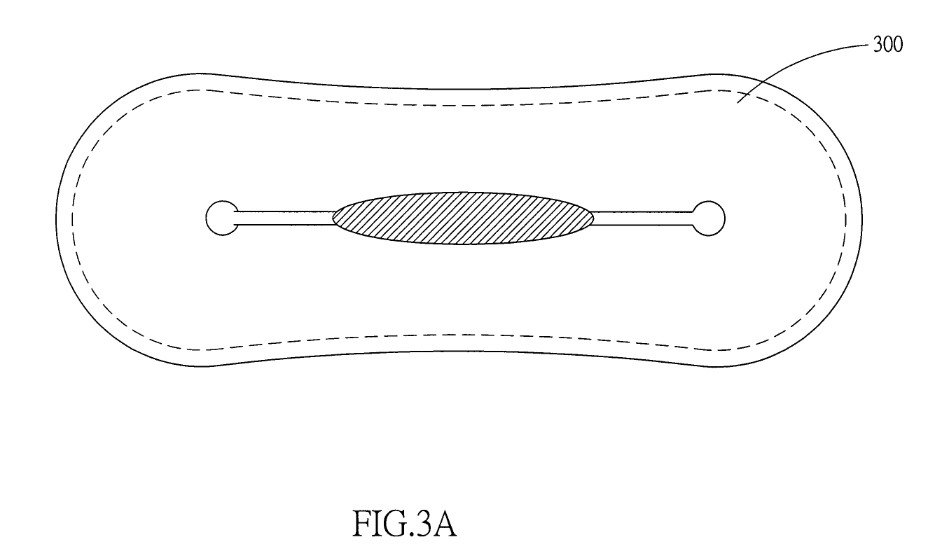

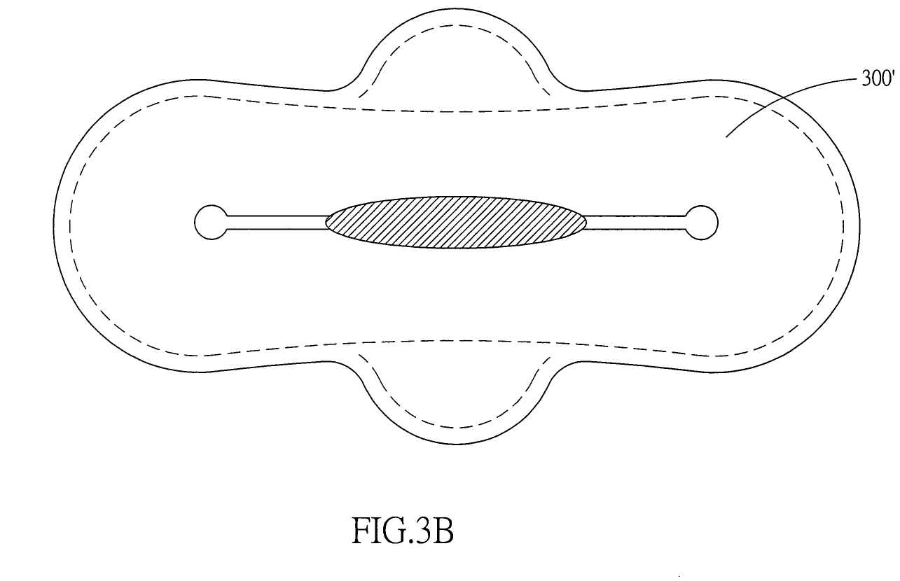

In another preferred example of this embodiment, the supporting layer 140 comprises two adhesive layers located above and below the supporting layer 140. The upper adhesive layer is used to fix the absorbing layer 120 onto the supporting layer 140, and the lower adhesive layer is used to fix the supporting layer 140 onto a noninvasive sanitary utensil, such as: sanitary napkin, pantyliner, or other nursing products. Additionally, the noninvasive collection device further comprises a protective sheet 160 outside the adhesive layer(s), so as to give facilities for portability and transportation. In practice, the user should remove the protective sheet 160, and then glue the supporting layer 140 onto an appropriate position of the noninvasive sanitary utensil, such as: 300 and 300′ as shown in FIG. 3A and FIG. 3B.

Because of the small size of the noninvasive collection device, the users would not feel uncomfortable when the noninvasive sanitary utensil combined with the noninvasive collection device and is used in a conventional manner in user's undergarment. By the way, when the noninvasive sanitary utensil combined with the noninvasive collection device is used, the absorbing layer collects bio-specimens, such as secretions, cells, and tissues from the genital regions. Furthermore, the noninvasive collection device can be stored for transportation and subsequent bio-analysis of diseases, such as: blood borne infections, sexually transmitted diseases, disease gene, and so on. Moreover, the provided noninvasive collection device can be applied in disease monitoring, gene research, or other technologies.

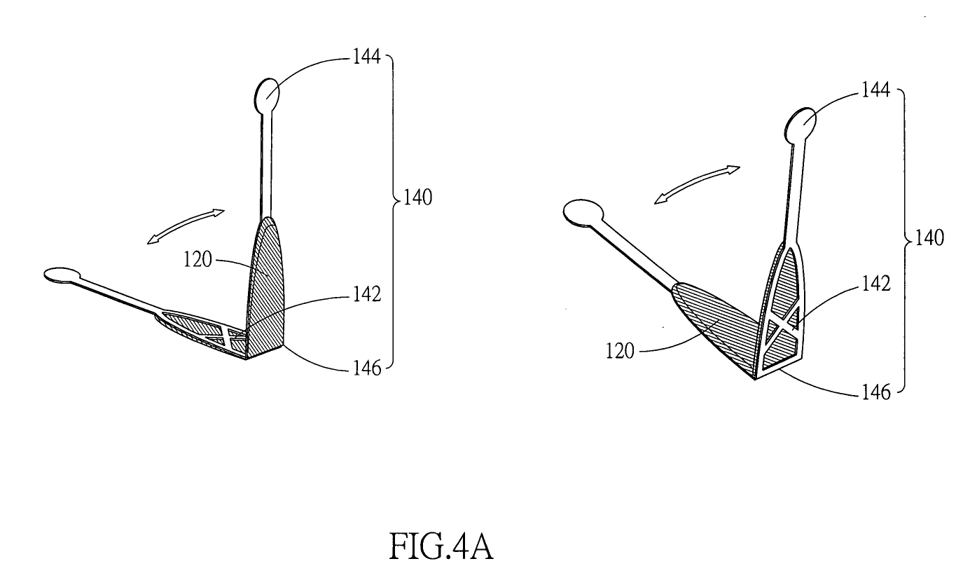

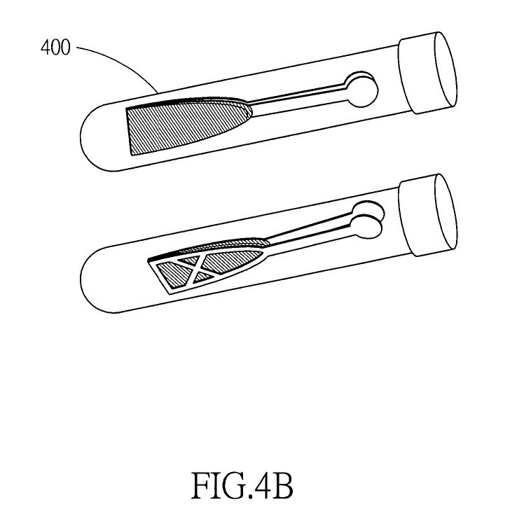

After self-collecting, the used collection device can be plucked from the noninvasive sanitary utensil by the pulling element 144. Next, the user can fold the plucked collection device by the folding line 146 located at the middle of the supporting layer 140, as shown in FIG. 4A. The folded collection device is then dried, and the drying methods comprise hot-air drying, or other drying methods which would not damage or change the bio-specimens. Additionally, referring to FIG. 4B, the dried noninvasive collection device is stored in containers, such as test tube 400, for further analysis.

Pelvic examinations and Pap smear are common medical technologies associated with female diseases. However, most Asian women find the Pap smear and pelvic examinations embarrassing, and false positive or false negative results of Pap smear can happen and caused by wrong judgment or incorrect sampling procedure. Therefore, new medical technology of female disease is developed to collect the secretion from the genital regions with further biochemical analysis. Nevertheless, the used conventional collecting means, such as: cotton ball, and pantyliner, are directly transferred to medical laboratories for disease detecting. The conventional collecting means not only result in inconvenience in transportation, but also cause problems in preservation.

Comparing to the conventional system, the present invention provides a removable noninvasive collection device which can be temporary fixed on a noninvasive sanitary utensil. According to this invention, the user can fix the noninvasive collection device onto a noninvasive sanitary utensil for collecting bio-specimens from the genital regions in menstrual cycle or in ordinary days. Furthermore, after self-collecting step, the used collection device can be plucked from the noninvasive sanitary utensil, then dried, and stored in a container for transportation and subsequent bio-analysis. Moreover, the provided noninvasive collection device can be applied in disease monitoring, gene research, or other technologies. To sum up, this invention provides an appropriate and complete collection device and method for medical technologies associated with female diseases.

Because the self-sampling procedure provokes less discomfort, pain, and embarrassment than pelvic examinations or Pap smear testing, the present invention provides a better option for disease screening. In the above preferred embodiments, the provided noninvasive collection device could decrease misgivings about discomfort or risks of infections, so that the noninvasive collection device can be more acceptable than conventional system. Furthermore, after self-collecting step, the used collection device can be stored in a container to avoid leakage of smell, and give facilities for transportation and subsequent analysis. Moreover, the used collection device can be further analyzed with biotechnology, whereupon the results of screenings are more accurate, and more medical screenings can be adopted.

To sum up, the present invention discloses a noninvasive collection device for collecting bio-specimens of female, which comprises a fixing layer to fix the noninvasive collection device onto a noninvasive sanitary utensil, a supporting layer located atop the fixing layer, and an absorbing layer located atop the supporting layer. The user can fix the noninvasive collection device onto a noninvasive sanitary utensil, and then the noninvasive sanitary utensil combined with the noninvasive collection device is used in a conventional manner in user's undergarment. The absorbing layer is used for collecting secretions from the genital regions. After self-collecting step, the used collection device can be plucked from the noninvasive sanitary utensil and stored in a container for transportation and subsequent analysis.

Although specific embodiments have been illustrated and described herein, it is obvious to those skilled in the art that many modifications of the present invention may be made without departing from what is intended to be limited solely by the appended claims.

1. A noninvasive collection device for collecting bio-specimens of female, comprising:
a supporting layer; and
an absorbing layer, wherein said absorbing layer is located atop said supporting layer.

2. The device according to claim 1, wherein said supporting layer further comprises a porous part.

3. The device according to claim 1, wherein said supporting layer further comprises at least one pulling element.

4. The device according to claim 3, wherein said supporting layer comprises two pulling elements, and said pulling elements are oppositely located at said supporting layer.

5. The device according to claim 1, wherein said supporting layer further comprises an adhesive layer located below said supporting layer.

6. The device according to claim 6, wherein the device further comprises a protective sheet outside said adhesive layer.

7. The device according to claim 1, wherein said supporting layer further comprises a folding line, and said folding line is located in the middle of said supporting layer.

8. The device according to claim 7, further comprising a container, whereupon the folded said supporting layer and folded said absorbing layer by the folding line are stored in said container.

9. The device according to claim 8, wherein said container further comprises a centrifuge tube.
